(19) United States
(12) Patent Application Publication
(10) Pub. No.: US 2005/0123497 A1
(43) Pub. Date: Jun. 9, 2005

(54) Controlled benefit agent delivery system

(76) Inventors: Robert Richard Dykstra, Cleves, OH (US); Lon Montgomery Gray, Florence, KY (US); Gregory Scot Miracle, Hamilton, OH (US); Lois Sana Gallon, Cincinnati, OH (US); Peter James Malton, Staines (GB)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/978,938

(22) Filed: Nov. 1, 2004

Related U.S. Application Data
(63) Continuation of application No. 10/225,428, filed on Aug. 22, 2002, now Pat. No. 6,870,210.
(60) Provisional application No. 60/345,469, filed on Oct. 19, 2001.

(51) Int. Cl. A61K 7/46; A61K 7/075; A61K 7/08

(52) U.S. Cl. 424/70.27; 512/1

(57) ABSTRACT

The present invention relates to a benefit agent delivery system that can, when directly applied to a substrate, provide a longer benefit term than when a benefit agent alone is applied to said substrate. Typical benefit agents include perfume raw materials such as perfume aldehydes and ketones.

### CROSS-REFERENCE TO RELATED APPLICATIONS

This application claims priority under 35 U.S.C. § 120 to U.S. patent application Ser. No. 10/225,428 filed Sep. 26, 2002, which in turn claims priority under 35 U.S.C. § 119(e) to U.S. Provisional Application Ser. No. 60/345,469 filed Oct. 19, 2001.

### FIELD OF THE INVENTION

The present invention relates to benefit agent delivery systems that, when directly applied to a substrate, provide a longer benefit term than the benefit agent alone.

### BACKGROUND OF THE INVENTION

It is frequently desirable or advantageous to treat the surfaces of a variety of substrates, for example fabrics, with benefit agents such as perfumes, flavors, pharmaceuticals and/or biocontrol agents including biocides, insecticides, mildewcides, and the like. Generally, the objective of such treatment is to leave deposited on the surfaces of the substrates enough benefit agent so that there is a residual benefit imparted to the substrate surface.

Products, systems and methods for depositing benefit agents onto the surfaces of substrates are known in the art. For example, in the context of fabric treatment, such as fabric laundering, a variety of products can be used to form benefit imparting aqueous washing liquors or rinse baths.

Other products that provide improved deposition onto substrate surfaces are benefit agents such as perfumes. Such products are described in PCT patent application Ser. Nos. WO 00/02991; WO 00/02981; WO 00/02987 and WO 00/02982. These patent publications disclose compositions wherein a residual benefit agent is realized by incorporating a reaction product formed from amine-based compounds and certain types of benefit agents that will react with such amine-based compounds into substrate treatment products.

However, notwithstanding the advances in the art, there remains a continuing need for benefit agent delivery systems that are especially effective for directly delivering residual and long-lasting benefit agents to substrates.

### SUMMARY OF THE INVENTION

The present invention relates to a benefit agent delivery system suitable for delivering a benefit agent to a substrate, wherein the benefit agent delivery system comprises a benefit agent and an amine comprising an amine moiety selected from the group consisting of primary amines, secondary amines and mixtures thereof, such that when said amine and said benefit agent are directly applied to a substrate, the benefit agent provides a benefit to the substrate for a longer period of time than when said amine is not present.

The present invention also relates to products comprising the aforementioned delivery system and methods of using same.

### DETAILED DESCRIPTION

Applicants' benefit delivery systems provide formulation flexibility as initial and ongoing benefit release levels can be adjusted to achieve almost any type of release profile, including profiles that are consistent with time. For example, in one aspect of Applicants' invention the amount of benefit agent that is initially released is perceptibly less than that normally released, however, such release rate is more consistent with time. In the event that a combination of high initial and sustained release is desired, the skilled artisan need only alter the level or mix of the one or more of benefit agents employed. In general, the benefit agent and amine component of the present invention should be in intimate contact for a sufficient period of time, before being applied, to provide the optimum benefit. While such contact period may vary from application to application, Applicants' have discovered that a seven day contact period is sufficient for most applications.

The components of the benefit agent delivery systems herein are selected and processed such that the resulting delivery systems are especially effective for delivering the benefit agent to a substrate that has been directly contacted with delivery system. Under such conditions, the benefit agent delivered to the substrate surface will provide its benefit thereto for a longer period of time than if no amine-based compound were present in the delivery system.

The benefit agent delivery systems of the present invention are particularly useful in various applications and/or products that are intended to be applied without dilution, such as fine fragrance perfume applications and/or products, home care perfume products, such as freshening compositions, that may comprise cyclodextrins, and that are typically applied to upholstery, carpets and other fabric articles, hard surface treating compositions, beauty care applications, such as creams, lotions, deodorants, antiperspirants, and other topical compositions, hair care compositions, such as hair spray, leave-in conditioners, and the like.

Definitions and Test Methods:

“Directly applied” and/or “delivering directly” as used herein means that a benefit agent is applied to a substrate via the benefit agent delivery system such that the benefit provided by the benefit agent is realized and/or recognized prior to dilution. For example, a benefit agent is sprayed onto a substrate and/or wiped on to a substrate, rather than having the benefit agent contact or deposit indirectly onto a substrate from a dilute solution (i.e., wash liquor).

The term “unit which can substitute for hydrogen” means “chemical moieties which can replace a hydrogen atom on a hydrocarbon chain, an aryl ring, and the like, or replace a hydrogen atom, two hydrogen atoms, or three hydrogen atoms from a carbon atom to form a moiety, or replace hydrogen atoms from adjacent carbon atoms to form a moiety.” For example, a substituted unit that requires a single hydrogen atom replacement includes halogen, hydroxyl, and the like. A two hydrogen atom replacement includes carbonyl, oximino, and the like. Three hydrogen replacement includes cyano, and the like.

The term “substituted” means that a moiety, inter alia, aromatic ring, alkyl chain, can have one or more of the hydrogen atoms replaced by a substituent. For example, 4-hydroxyphenyl is a “substituted aromatic carbocyclic ring”, and 3-guanidinopropyl is a “substituted C₃ alkyl unit.”

The term “hydrocarbyl” means “any unit which comprises carbon and hydrogen atoms, whether linear, branched, cyclic, and regardless of how many of the hydrogen atoms are substituted with a suitable “substituted” unit.” Non-limiting examples of “hydrocarbyl” units include methyl, benzyl, 6-hydroxyoctanyl, m-chlorophenyl, 2-(N-methylamino)propyl, and the like. The following are non-limiting examples of moieties, which can replace hydrogen atoms on carbon to form a “substituted hydrocarbyl” unit:
i) —NHCOR³⁰;
ii) —COR³⁰;
iii) —COOR³⁰;
iv) —COCH═CH₂;
v) —C(═NH)NH₂;
vi) —N(R³⁰)₂;
vii) —NHC₆H₅;
viii) ═CHC₆H₅;
ix) —CON(R³⁰)₂;
x) —CONHNH₂;
xi) —NHCN;
xii) —OCN;
xiii) —CN;
xiv) F, Cl, Br, I, and mixtures thereof;
xv) ═O;
xvi) —OR³⁰ ;
xvii) —NHCHO;
xviii) —OH;
xix) —NHN(R³⁰)₂;
xx) ═NR³⁰;
xxi) ═NOR³⁰;
xxii) —NHOR³⁰;
xxiii) —CNO;
xxiv) —NCS;
xxv) ═C(R ³⁰)₂;
xxvi) —SO₃M;
xxvii) —OSO₃M;
xxviii) —SCN;
xxix) —P(O)H₂;
xxx) —PO₂;
xxxi) —P(O)(OH)₂;
xxxii) —SO₂NH₂;
xxxiii) —SO₂R³⁰;
xxxiv) —NO₂;
xxxv) —CF₃, —CCl₃, —CBr₃;
xxxvi) and mixtures thereof;
wherein R³⁰ is hydrogen, C₁-C₂₀ linear or branched alkyl, C₆-C₂₀ aryl, C₇-C₂₀ alkylenearyl, and mixtures thereof; M is hydrogen, or a salt forming cation. Suitable salt forming cations include, sodium, lithium, potassium, calcium, magnesium, ammonium, and the like. Non-limiting examples of an alkylenearyl unit include benzyl, 2-phenylethyl, 3-phenylpropyl, 2-phenylpropyl.

The term “inorganic carrier”, means a carrier that comprises of non- or substantially non-carbon based backbones.

Odor Intensity Index Method: Odor Intensity Index is a value determined by expert graders who evaluate test chemicals for odor when such the pure chemicals are diluted at 1% in dipropylene glycol (DPG), odor-free solvent used in perfumery. This concentration percentage is representative of typical usage levels. Smelling strips, or so called “blotters”, are dipped in test solutions and presented to expert panelists for evaluation. Expert panelists are assessors trained for at least six months in odor grading and whose gradings are checked for accuracy and reproducibility versus a reference on an on-going basis. For each amine compound, a panelist is presented two blotters: one reference (Me Anthranilate, unknown from the panelist) and the test sample. The panelist is asked to rank both smelling strips on the 0-5 odor intensity scale, 0 being no odor detected, 5 being very strong odor present.

The following represents Odor Intensity Index of some amine compounds suitable for use in the present invention. In each case, numbers are arithmetic averages among 5 expert panelists and the results are statistically significantly different at 95% confidence level:

| | |
|---|---|
| Methylanthranilate 1% (reference) | 3.4 |
| Ethyl-4-aminobenzoate (EAB) 1% | 0.9 |
| 1,4-bis-(3-aminopropyl)-piperazine (BNPP) 1% | 1.0 |

Protocol 1.1—Longevity Test: Each benefit delivery system that comprises a perfume raw material and an amine, is tested in accordance with the instant protocol. Each perfume aldehyde or ketone (P) found in such perfume raw material is tested with each amine to determine if the combination (PA) demonstrates a longevity that is greater than that obtained for P alone.

Multiple benefit agents may be tested together, at the same time, in the presence of multiple amines, as long as the analytical measurements are not compromised by such combination. By way of illustration, a benefit delivery system that contains six perfumes—three of which are aldehyde or a ketone perfumes (P¹, P₂ and P³), and three of which are not aldehyde or ketone perfumes, and a single amine (A¹) requires the following single-variable test: (P¹A¹, P²A¹ and P³A¹) verses (P , P and p³), provided that said benefit agents are chromatographically separable such that the amount of each perfume aldehyde or ketone is easily determined in the presence of the other. Perfume aldehydes or ketones that are not chromatographically separable from one another must be run in separate tests. If, for example, P¹ and P³ are not separable, then one of the following sets of tests is required:
I. (P¹A¹ and P²A¹) vs. (P¹ and P²), and (P³A¹) vs. (P³); or
II. (P²A¹ and P³A¹) vs. (P² and P³), and (P¹A¹) vs. (P¹); or
III. (P¹A¹) vs. (P¹), and (P²A¹) vs. (P²), and (P³A¹) vs. (P³).
   No P in any test should be present at a concentration greater than ten times the concentration of another P in the same test. In such a case, separate tests are indicated.
a.) Determination of the Concentration of Benefit Agent(s) and Amine in the Test Solution
The absolute concentration for the test solution (TS) to be used in a Benefit Agent Longevity Test (LT) is determined as follows.
The perfume aldehyde(s) or ketone(s) and amine(s) that are to be tested together are dissolved in 50:50 (v/v) ethanol:water at a concentration equal to that used in the benefit agent delivery system. The solution is closed to the atmosphere and aged for 24 hours at room temperature to obtain the initial test solution, designated TS₀. A 1.0 mL aliquot of TS₀ is pipetted onto a 4 cm diameter circle weighing 0.45-0.65 g (weight of circles in a given test should be the same ±0.02 g) cut from an 86/14 cotton/poly terry wash cloth (obtained from EMC, 7616 Reinfold Drive, Cincinnati, Ohio 45237). The cloth, charged with test solution, is left open to the atmosphere under ambient conditions and subsequently analyzed via headspace gas chromatography (HSGC) to determine the amount of each perfume aldehyde or ketone in the headspace at each of the following times: 0.50, 1, 2, 4, 6, 8, and 24 hours.
The absolute concentration of perfume aldehyde(s) or ketone(s) and amine(s) to be used in the LT is the lowest concentration in a series of solutions based on TS₀ at which each perfume aldehyde or ketone in the TS is detected by HSGC at no less than one of the designated time points. If this condition is not met by TS₀, the concentration of the test solution is doubled and the new solution (TS₁) is tested in the same manner. The process is repeated until the condition is met. The concentration of perfume aldehyde(s) or ketone(s) and amine(s) in the test solution that meets the expressed condition (TSₙ) is related to the concentration of the perfume aldehyde(s) or ketone(s) and amine(s) in TS₀ according to the following equation:
   [P, A] in TSₙ=2ⁿ·[P, A] in TS₀; where n=0, 1, 2, 3 . . .
b.) Headspace Gas Chromatography
   Equipment required consists of:
      1.) A trap containing a porous polymer having the ability to retain aroma materials, preferably Tenax TA 35/60 mesh.
      2.) A source of pure helium.
      3.) A headspace collector to contain the fabric circle and allow benefit agent to partition into the vapor headspace and reach equilibrium.
      4.) GC-MS with headspace capabilities.
   Suitable equipment is as referenced in S. Maeno, P. A. Rodriguez. J. Chromatography, A731(1996) 201-215. It consists of equipment to transfer the equilibrated headspace vapors containing perfume aldehydes or ketones, which have been captured on a porous polymer, onto a GC for quantitative analysis. This equipment is able to heat the porous polymer trap containing the collected headspace, transferring the vapors to a cold trap cooled to ←100° C. (generally by liquid nitrogen). Following complete transfer to the cold trap, the cold trap is flash heated in a short period of time—typically about 1 minute—to a temperature of approximately 0° C. followed by a normal temperature gradient to about 280° C., resulting in the transfer of the headspace vapors directly onto a capillary GC column. A typical column is a 30-60 meter long with an i.d. of 0.18-0.32 mm, with a stationary phase composed of 100% dimethylpolysiloxane or (5%-phenyl)-methylpolysiloxane. The GC has the capability of quantitating and identifying said perfume aldehydes or ketones. Identification is accomplished via Mass Spectrometry and quantification is performed using a separate detector, such as an FID (flame ionization) or PID (photo ionization) detector.
c.) Longevity Test
   A given test solution TSₙ meeting the condition described above is prepared. A second test solution TS_{c} is prepared containing all the components of TSₙ at exactly the same concentrations as in TSₙ except that any amines have been removed. TS_{c} serves as the control solution in the test.
   Data is gathered for a given test solution (either TS_{c} or TSₙ) as follows: A 1.0 mL aliquot of TS is pipetted onto a 4 cm diameter circle cut from an 86/14 cotton/poly terry wash cloth. The cloth charged with TS is left open to the atmosphere under ambient conditions and subsequently analyzed via headspace gas chromatography (HSGC) to determine the amount of each benefit agent in the headspace at each of the following seven designated times: 0.50, 1, 2, 4, 6, 8, and 24 hours.
   The conditions used for the analysis in each case are identical. The only difference for the two sets of data is that one set is obtained from a solution containing amine and the other set is obtained from a solution not containing amine.
   A longevity benefit is confirmed for a particular P when the quantitative amount of the P in the headspace from TSₙ at any one of the seven designated times points is greater (statistically significant at 95% confidence) than the amount of the same P in the headspace from TS_{c} at the corresponding time point.
d.) Example Results

The following table demonstrates the type of results that can be obtained from a Longevity Test. The data confirms a longevity benefit for P¹ (at t=4 h, the area count from TSₙ>TS_{c}) and P² (at t=1 h, the area count from TSₙ>TS_{c}) in the presence of A¹.

| | HSGC Area Count for Benefit Agent with and without A¹ | | | | | |
|---|---|---|---|---|---|---|
| | P¹ | | P² | | P³ | |
| Time (h) | TS_{c} | TSₙ | TS_{c} | TSₙ | TS_{c} | TSₙ |
| 0.50 | 12000 | 1000 | 8000 | 2000 | 30000 | 26000 |
| 1 | 6000 | 1500 | 400 | 1500 | 5000 | 4500 |
| 2 | 3000 | 2500 | 20 | 1000 | 850 | 700 |
| 4 | 1500 | 4000 | ND | 500 | 140 | 90 |
| 6 | 750 | 1500 | ND | 150 | 25 | ND |
| 8 | 220 | 500 | ND | 30 | ND | ND |
| 24 | ND | 50 | ND | ND | ND | ND |

ND = Not detected.

Amine

Applicants discovered that non-aromatic amines provide Applicants' delivery system with especially effective and efficient release characteristics. While not being bound by theory, Applicants believe that such characteristics are due to the substantial difference in the pKa of aromatic and non-aromatic amines. However, in general, suitable amines include mono-amines, such as a hydroxyamine or a polyamine so long as its molecular weight is greater than about 50 Daltons and so long as at least about 10% of its amino moieties are selected from the group consisting of primary amines, secondary amines and mixtures thereof. In one aspect of Applicants' invention, the amine comprises a primary amine moiety, and in another aspect the amine comprises, based on the total number of amine moieties in the amine, from about 10% to 100% primary amine moieties. In another aspect of Applicants' invention the amine comprises, based on the total number of amine moieties in the amine, from about 15% to 100% primary amine moieties. Suitable hydroxyamines include hydroxyamines that have an average molecular weight of greater than about 100 g/mole. Specific examples of such hydroxyamines include hydroxyamines selected from the group consisting of 2-hydroxyamines, 3-hydroxyamines and mixtures thereof. Suitable polyamines include polyamines that have an average molecular weight of from about 100 to about 2.10×10⁶ g/mol. In another aspect of Applicants' invention, suitable amines include amines having an Odor Intensity Index of less than that of a 1% solution of methylanthranilate in dipropylene glycol.

A general structure for suitable primary amines is as follows:
B—(NH₂)ₙ;
wherein B is a carrier material, and n is an index of value of at least 1. Compounds containing a secondary amine group have a structure similar to the above with the exception that the compound comprises one or more —NH— groups as well as —NH₂ groups. In one aspect of Applicants' invention, the amine compound, such as certain volatile amines, will not impart a sticky feel or undesired residue to a substrate that is treated with Applicants' invention.

The hydroxy amines of the present invention have the general formula: 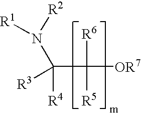
wherein, R¹-R⁶ units can be any substituted or unsubstituted hydrocarbyl unit, non-limiting examples include:
a) hydrogen;
b) C₁-C₁₀ substituted or unsubstituted linear alkyl;
c) C₃-C₁₀ substituted or unsubstituted branched alkyl;
d) C₂-C₁₀ substituted or unsubstituted linear alkenyl; as in the case of α, β, γ,
e) C₃-C₁₀ substituted or unsubstituted branched alkenyl;
f) C₃-C₁₅ substituted or unsubstituted cycloalkyl;
g) C₄-C₁₅ substituted or unsubstituted branched cycloalkyl;
h) C₄-C₁₅ substituted or unsubstituted cycloalkenyl;
i) C₅-C₁₅ substituted or unsubstituted branched cycloalkenyl;
j) C₆-C₁₅ substituted or unsubstituted aryl;
k) C₆-C₂₂ substituted or unsubstituted heterocyclicalkyl;
l) C₆-C₂₂ substituted or unsubstituted heterocyclicalkenyl;
m) and mixtures thereof;
   alternatively the R³-R⁶ units can be taken together to form a substituted or unsubstituted ring having in the ring from 3 to 10 carbon atoms; for example, R³ and R⁵ taken together can be fused ring comprising ketones. In one aspect of Applicants' invention, the index m is an integer from 1 to 3.

For the present invention R⁷is independently selected from any substituted or unsubstituted hydrocarbyl unit, non-limiting embodiments are selected from the group consisting of:
a) R⁶;
b) hydroxyl;
c) a carbonyl comprising unit having the formula:
   —(CH₂)ₓCOR⁸
   wherein R⁸ is:
   i) —OH;
   ii) —OR⁹ wherein R⁹ is hydrogen, C₁-C₁₅ substituted linear alkyl, C₁₁-C₁₅ unsubstituted linear alkyl, C₁-C₁₅ substituted branched alkyl, C₁₁-C₁₅ unsubstituted branched alkyl, C₂-C₂₂ substituted or unsubstituted linear alkenyl, C₃-C₂₂ substituted or unsubstituted branched alkenyl, or mixtures thereof, wherein said substitution is not halogen or thioalkyl; R⁹ is methyl, R⁹ is hydrogen and Z is oxygen or sulfur when an oxazolidine is formed from the methyl esters of serine, threonine, cysteine, and the like;
   iii) —N(R¹⁰)₂ wherein R¹⁰ is hydrogen, C₁-C₆ substituted or unsubstituted linear alkyl, C₃-C₆ substituted or unsubstituted branched alkyl, or mixtures thereof;
   iv) C₁-C₂₂ substituted or unsubstituted linear alkyl;
   v) C₁-C₂₂ substituted or unsubstituted branched alkyl;
   vi) C₂-C₂₂ substituted or unsubstituted linear alkenyl;
   vii) C₃-C₂₂ substituted or unsubstituted branched alkenyl;
   viii) C₃-C₂₂ substituted or unsubstituted cycloalkyl;
   ix) C₆-C₂₂ substituted or unsubstituted aryl;
   x) C₆-C₂₂ substituted or unsubstituted heterocyclicalkyl;
   xi) C₆-C₂₂ substituted or unsubstituted heterocyclicalkenyl;
   the index x is from 0 to 22;
d) alkyleneoxy units having the formula:
   —[C(R¹¹)₂]_{y}[C(R¹¹)₂C(R¹¹)₂O]_{z}R¹¹
   wherein each R¹¹ is independently;
   i) hydrogen;
   ii) —OH;
   iii) C₁-C₄ alkyl;
   iv) or mixtures thereof;
   two R¹¹ units can be taken together to form a C₃-C₆ spiroannulated ring, carbonyl unit, or mixtures thereof; y has the value from 0 to 10, z has the value from 1 to 50;
e) and mixtures thereof.
   In one aspect of Applicants' invention, R² and R⁷ are hydrogen. In another aspect of Applicants' invention, R¹ is selected from hydrogen and C₁-C₁₀ linear or branched alkyl. Non-limiting examples of such amines wherein m is 1 are represented by the following formula:

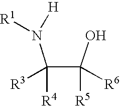

Non-limiting examples of suitable hydroxyamines include:

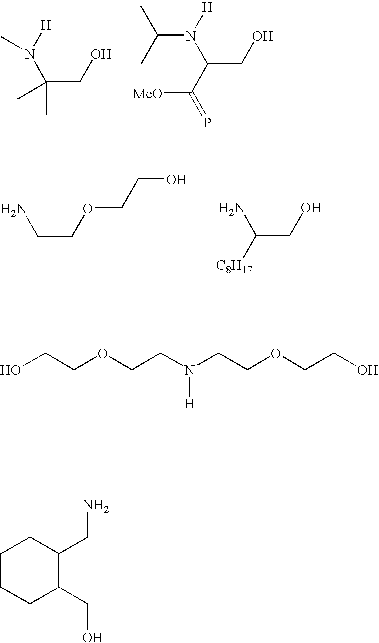

Suitable B carriers include both inorganic and organic carrier moieties. Primary amines, utilizing inorganic carriers, are those selected from mono or polymers or organic-organosilicon copolymers of amino derivatised organo silane, siloxane, silazane, alumane, aluminum siloxane, or aluminum silicate compounds. Typical examples of such carriers are: organosiloxanes with at least one primary amine moiety like the diaminoalkylsiloxane [H₂NCH₂(CH₃)₂Si]O, or the organoaminosilane (C₆H₅)₃SiNH₂ described in: Chemistry and Technology of Silicone, W. Noll, Academic Press Inc. 1998, London, pp 209, 106).

Primary amines, utilizing organic carriers, may be selected from aminoaryl derivatives, polyamines, amino acids and derivatives thereof, substituted amines and amides, glucamines, dendrimers, polyvinylamines and derivatives thereof, and/or copolymers thereof, alkylene polyamine, polyaminoacid and copolymers thereof, cross-linked polyaminoacids, amino substituted polyvinylalcohol, polyoxyethylene bis amine or bis aminoalkyl, aminoalkyl piperazine and derivatives thereof, bis (amino alkyl) alkyl diamine linear or branched, and mixtures thereof. Suitable aminoaryl derivatives are the amino-benzene derivatives including the alkyl esters of 4-amino benzoate compounds. In one aspect of Applicants' invention, suitable aminoaryl derivatives are selected from the group consisting of ethyl-4-amino benzoate, phenylethyl-4-aminobenzoate, phenyl-4-aminobenzoate, 4-amino-N′-(3-aminopropyl)-benzamide, and mixtures thereof.

Suitable polyamines include polyethyleneimines polymers, poly[oxy(methyl-1,2-ethanediyl)], α-(2-aminomethylethyl)-ω-(2-aminomethyl-ethoxy)-(=C.A.S No. 9046-10-0); poly[oxy(methyl-1,2-ethanediyl)], α-hydro-)-ω-(2-aminomethylethoxy)-, ether with 2-ethyl-2-(hydroxymethyl)-1,3-propanediol (=C.A.S. No. 39423-51-3); commercially available from Huntsman Performance Chemicals of Houston, Tex., USA under the tradename Jeffamines® T-403, D-230, D-400, D-2000; 2,2′,2″-triaminotriethylamine; 2,2′-diamino-diethylamine; 3,3′-diamino-dipropylamine, 1,3 bis aminoethyl-cyclohexane commercially available from Mitsubishi Chemical Corporation, of 5-2, Marunouchi2-Chome, Chiyoda-ku, Tokyo 100-0005 and the C12 Sternamines commercially available from Clariant International Ltd, Rothausstrasse 61 CH-4132 Muttenz 1/Schweiz like the C₁₂ Sternamin(propylenamine)ₙ with n=3/4, and mixtures thereof. In one aspect of Applicants' invention, the polyamines are polyethyleneimines commercially available from BASF Corporation 3000 Continental Drive-North Mount Olive, N.J. 07828-1234 under the tradename Lupasol® like Lupasol® FG (MW 800), G20wfv (MW 1300), PR8515 (MW 2000), WF (MW 25000), FC (MW 800), G20 (MW 1300), G35 (MW 1200), G100 (MW 2000), HF (MW 25000), P (MW 750000), PS (MW 750000), SK (MW 2000000), SNA (MW 1000000). In another aspect of Applicants' invention such polyamines include Lupasol® HF or WF (MW 25000), P (MW 750000), PS (MW 750000), SK (MW 2000000), 620wfv (MW 1300) and PR 1815 (MW 2000).

Suitable amino acids for use herein may be selected from tyrosine, tryptophane, lysine, glutamic acid, glutamine, aspartic acid, arginine, asparagine, phenylalanine, proline, glycine, serine, histidine, threonine, methionine, and mixtures thereof. In one aspect of Applicants' invention suitable amino acids are selected from tyrosine, tryptophane, and mixtures thereof. In another aspect of Applicants' invention, amino acid derivatives are selected from tyrosine ethylate, glycine methylate, tryptophane ethylate, and mixtures thereof.

Suitable amines and amides for use herein may be selected from nipecotamide, N-coco-1,3-propenediamine; N-oleyl-1,3-propenediamine; N-(tallow alkyl)-1,3-propenediamine; 1,4-diamino cyclohexane; 1,2-diamino-cyclohexane; 1,12-diaminododecane, and mixtures thereof.

Other primary amine compounds suitable for use herein include glucamines. In one aspect of Applicants' invention, said glucamines are selected from 2,3,4,5,6-pentamethoxy-glucamine; 6-acetylglucamine, glucamine, and mixtures thereof.

Other suitable amine compounds include the polyethylenimine and/or polypropylenimine dendrimers and the commercially available Starburst® polyamidoamines (PAMAM) dendrimers, generation G0-G10 from Dendritech Inc. of Dendritech, Inc. Midland, Mich. U.S.A. and the dendrimers Astramols®, generation 1-5 from DSM of Geleen, The Netherlands said dendrimers being DiAminoButane PolyAmine DAB (PA)x dendrimers with x=2ⁿ×4 and n being generally being between 0 and 4.

Suitable polyamino acids may contain alanine, serine, aspartic acid, arginine, valine, threonine, glutamic acid, leucine, cysteine, histidine, lysine, isoleucine, tyrosine, asparagine, methionine, proline, tryptophan, phenylalanine, glutamine, glycine or mixtures thereof. In one aspect of Applicants' invention, the required amine comprises a polyamino acid selected from the group consisting of polylysine, polyarginine, polyglutamine, polyasparagine, polyhistidine, polytryptophane or mixtures thereof. In another aspect of Applicants' invention, the required amine comprises polylysine or polyamino acids where more than 50% of the amino acids are lysine. In another aspect of Applicants' invention, the amine comprises a polyamino acid having a molecular weight of 500 to 10,000,000 Da. In another aspect of Applicants' invention, the amine comprises a polyamino acid having a molecular weight of between 2,000 and 25,000 Da. Examples and supply of polyaminoacids containing lysine, arginine, glutamine, asparagine are given in the Bachem 1996, Peptides and Biochemicals catalog. For example polylysine can be supplied as polylysine hydrobromide. Polylysine hydrobromide is commercially available from Sigma, Applichem, Bachem and Fluka.

Suitable amines also include ethoxylated polylysine, provided a requisite amount of primary amino groups remains in the polymer; crosslinked polyamino acids and co-polymerized polyamino acids and amino acids. Although crosslinked polyamino acids are useful, such acids need to have free primary and/or secondary amino groups. In one aspect of Applicants' invention, the amine comprises a crosslinked polyamino acid having a molecular weight of 20,000 to 10,000,000 Da. In another aspect of Applicants' invention, the amine comprises a crosslinked polyamino acid having a molecular weight of between 200,000 and 2,000,000 Da.

Suitable co-polymerized polyamino acids and amino acids may be co-polymerized with acids, amides, acyl chlorides such as aminocaproic acid, adipic acid, ethylhexanoic acid, caprolactam or mixtures thereof. The molar ratio used in these copolymers typically ranges from 1:1 (reagent/amino acid (lysine)) to 1:20, or from 1:1 to 1:10.

Non-limiting examples of suitable chemically modified amino acids include benzyloxycarbonyl, aminobutyric acid, butyl ester, pyroglutamic acid. More examples of common modifications of amino acids and small amino acid fragments can be found in the Bachem, 1996, Peptides and Biochemicals Catalog. In chemically modified amino acids, the amine or acidic function of the amino acid has typically reacted with a chemical reagent.

Non-limiting examples of suitable amino functional polymers that contain at least one primary amine group include:
Polyvinylamine with a MW of about 300-2.10E6 Da;
Polyvinylamine alkoxylated with a MW of about 600, 1200 or 3000 Da and an ethoxylation degree of 0.5;
Polyvinylamine vinylalcohol-molar ratio 2:1, polyvinylaminevinylformamide-molar ratio 1:2 and polyvinylamine vinylformamide-molar ratio 2: 1;
Triethylenetetramine, diethylenetriamine, tetraethylenepentamine;
Bis-aminopropylpiperazine;
Polyamino acid (L-lysine/lauric acid in a molar ratio of 10/1), Polyamino acid (L-lysine/aminocaproic acid/adipic acid in a molar ratio of 5/5/1), ), Polyamino acid (L-lysine/aminocaproic acid/ethylhexanoic acid in a molar ratio of 5/3/1) Polyamino acid (polylysine-cocaprolactam); Polylysine; Polylysine hydrobromide; cross-linked polylysine,
amino substituted polyvinylalcohol with a MW ranging from 400 Da to 300,000 Da;
polyoxyethylene bis [amine] available from e.g. Sigma;
polyoxyethylene bis [6-aminohexyl] available from e.g. Sigma;
N,N′-bis-(3-aminopropyl)-1,3-propanediamine linear or branched (TPTA);
N,N′-bis-(3-aminopropyl)ethylenediamine; and
1,4-bis-(3-aminopropyl) piperazine (BNPP).

In one aspect of Applicants' invention, amines are selected from ethyl-4-amino benzoate, polyethyleneimine polymers commercially available from BASF Corporation 3000 Continental Drive-North Mount Olive, N.J. 07828-1234 under the tradename Lupasol® like Lupasol® HF, P, PS, SK, SNA, WF, G20wfv and PR8515; the diaminobutane dendrimers Astramol®, polylysine, cross-linked polylysine, N,N′-bis-(3-aminopropyl)-1,3-propanediamine linear or branched; 1,4-bis-(3-aminopropyl) piperazine, and mixtures thereof. In another aspect of Applicants' invention, amines are selected from ethyl-4-amino benzoate, polyethyleneimine polymers having a molecular weight grater than 200 Da including those commercially available from BASF Corporation 3000 Continental Drive-North Mount Olive, N.J. 07828-1234 under the tradename Lupasol® like Lupasol® HF, P, PS, SK, SNA, WF, G20wfv and PR8515; polylysine, cross-linked polylysine, N,N′-bis-(3-aminopropyl)-1,3-propanediamine linear or branched, 1,4-bis-(3-aminopropyl) piperazine, and mixtures thereof.

Benefit Agent

Another essential component of the benefit agent delivery systems herein is a benefit agent. The benefit agents essentially used to form the delivery systems of this invention must be in the form of an aldehyde or ketone. It is understood that the genus of ketones includes those ketones, such as damascone, that comprise enone moieties. Such benefit agent can, for example, be selected from a flavor ketone or aldehyde, a pharmaceutical ketone or aldehyde, a biocontrol ketone or aldehyde, a perfume ketone or aldehyde and mixtures thereof. Perfume ketones and aldehydes are the most typical benefit agent used in Applicants' invention. A typical disclosure of suitable ketone and/or aldehydes, traditionally used in perfumery, can be found in “perfume and Flavor Chemicals”, Vol. I and II, S. Arctander, Allured Publishing, 1994, ISBN 0-931710-35-5. This publication is also incorporated herein by reference.

The perfume ketones utilized in the benefit agent delivery systems herein can comprise any material which is chemically a ketone and which can impart a desirable odor or freshness benefit to surfaces. The perfume ketone component can, of course, comprise more than one ketone, i.e., mixtures of ketones. In one aspect of Applicants' invention, the perfume ketone is selected from the group consisting of buccoxime; iso jasmone; methyl beta naphthyl ketone; musk indanone; tonalid/musk plus; Alpha-Damascone, Beta-Damascone, Delta-Damascone, Iso-Damascone, Damascenone, Damarose, Methyl-Dihydrojasmonate, Menthone, Carvone, Camphor, Fenchone, Alpha-Ionone, Beta-Ionone, dihydro-Beta-Ionone, Gamma-Methyl so-called Ionone, Fleuramone, Dihydrojasmone, Cis-Jasmone, Iso-E-Super® (1,2,3,4,5,6,7,8-octahydro-1,1,6,7-tetramethyl naphthalene-7-acetyl), Methyl- Cedrenyl-ketone or Methyl-Cedrylone, Acetophenone, Methyl-Acetophenone, Para-Methoxy-Acetophenone, Methyl-Beta-Naphtyl-Ketone, Benzyl-Acetone, Benzophenone, Para-Hydroxy-Phenyl-Butanone, Celery Ketone or Livescone, 6-Isopropyldecahydro-2-naphtone, Dimethyl-Octenone, Freskomenthe, 4-(1-Ethoxyvinyl)-3,3,5,5,-tetramethyl-Cyclohexanone, Methyl-Heptenone, 2-(2-(4-Methyl-3-cyclohexen-1-yl)propyl)-cyclopentanone, 1-(p-Menthen-6(2)-yl)-1-propanone, 4-(4-Hydroxy-3-methoxyphenyl)-2-butanone, 2-Acetyl-3,3-Dimethyl-Norbornane 6,7-Dihydro-1,1,2,3,3-Pentamethyl-4(5H)-Indanone, 4-Damascol, Dulcinyl or Cassione, Gelsone, Hexalon, Isocyclemone E, Methyl Cyclocitrone, Methyl-Lavender-Ketone, Orivon, Para-tertiary-Butyl-Cyclohexanone, Verdone, Delphone, Muscone, Neobutenone, Plicatone, Veloutone, 2,4,4,7-Tetramethyl-oct-6-en-3-one, Tetrameran, hedione, and mixtures thereof. While in another aspect of Applicants' invention the perfume ketones is selected from the group consisting of Alpha Damascone, Delta Damascone, Iso Damascone, Carvone, Gamma-Methyl-Ionone, Iso-E-Super® (1,2,3,4,5,6,7,8-octahydro-1,1,6,7-tetramethyl naphthalene-7-acetyl), 2,4,4,7-Tetramethyl-oct-6-en-3-one, Benzyl Acetone, Beta Damascone, Damascenone, methyl dihydrojasmonate, methyl cedrylone, hedione, and mixtures thereof.

Suitable perfume aldehydes can comprise any perfume material that is chemically an aldehyde, which can, like the perfume ketone component, impart a desirable odor or freshness benefit to surfaces. As with the perfume ketone benefit agents, the perfume aldehyde benefit agent component can comprise a single individual aldehyde or mixtures of two or more perfume aldehydes. Suitable perfume aldehyde materials for use in the delivery systems herein, whether by themselves or as part of a perfume aldehyde mixture, include melonal (2,6-Dimethyl-5-heptenal), triplal® (3-cyclohexene-1-carboxaldehyde, 2,4-dimethyl), Lugustral, adoxal; anisic aldehyde; cymal; ethyl vanillin; florhydral® (3-(3-Isopropyl-phenyl)-butyraldehyde); helional; heliotropin; hydroxycitronellal; koavone; lauric aldehyde; lyral® (4-(4-hydroxy-4-methyl pentyl)-3-cyclohexene-1-carboxaldehyde); methyl nonyl acetaldehyde; P. T. bucinal; phenyl acetaldehyde; undecylenic aldehyde; vanillin; 2,6,10-trimethyl-9-undecenal, 3-dodecen-1-al, alpha-n-amyl cinnamic aldehyde, 4-methoxybenzaldehyde, benzaldehyde, 3-(4-tert butylphenyl)-propanal, 2-methyl-3-(para-methoxyphenyl propanal, 2-methyl-4-(2,6,6-trimethyl-2(1)-cyclohexen-1-yl) butanal, 3-phenyl-2-propenal, cis-/trans-3,7-dimethyl-2,6-octadien-1-al, 3,7-dimethyl-6-octen-1-al, [(3,7-dimethyl-6-octenyl)oxy] acetaldehyde, 4-isopropylbenzyaldehyde, 1,2,3,4,5,6,7,8-octahydro-8,8-dimethyl-2-naphthaldehyde, 2,4-dimethyl-3-cyclohexen-1-carboxaldehyde, 2-methyl-3-(isopropylphenyl)propanal, 1-decanal; decyl aldehyde, 2,6-dimethyl-5-heptenal, 4-(tricyclo[5.2.1.0(2,6)]-decylidene-8)-butanal, octahydro-4,7-methano-1H-indenecarboxaldehyde, 3-ethoxy-4-hydroxy benzaldehyde, para-ethyl-alpha, alpha-dimethyl hydrocinnamaldehyde, alpha-methyl-3,4-(methylenedioxy)-hydrocinnamaldehyde, 3,4- methylenedioxybenzaldehyde, alpha-n-hexyl cinnamic aldehyde, m-cymene-7-carboxaldehyde, alpha-methyl phenyl acetaldehyde, 7-hydroxy-3,7-dimethyl octanal, Undecenal, 2,4,6-trimethyl-3-cyclohexene-1-carboxaldehyde, 4-(3)(4-methyl-3-pentenyl)-3-cyclohexen-carboxaldehyde, 1-dodecanal, 2,4-dimethyl cyclohexene-3-carboxaldehyde, 4-(4-hydroxy-4-methyl pentyl)-3-cylohexene-1-carboxaldehyde, 7-methoxy-3,7-dimethyloctan-1-al, 2-methyl undecanal, 2-methyl decanal, 1-nonanal, 1-octanal, 2,6,10-trimethyl-5,9-undecadienal, 2-methyl-3-(4-tertbutyl)propanal, dihydrocinnamic aldehyde, 1-methyl-4-(4-methyl-3-pentenyl)-3-cyclohexene-1-carboxaldehyde, 5 or 6 methoxy0hexahydro-4,7-methanoindan-1 or 2-carboxaldehyde, 3,7-dimethyloctan-1-al, 1-undecanal, 10-undecen-1-al, 4-hydroxy-3-methoxy benzaldehyde, 1-methyl-3-(4-methylpentyl)-3-cyclhexenecarboxaldehyde, 7-hydroxy-3,7-dimethyl-octanal, trans-4-decenal, 2,6-nonadienal, para-tolylacetaldehyde; 4-methylphenylacetaldehyde, 2-methyl-4-(2,6,6-trimethyl-1-cyclohexen-1-yl)-2-butenal, ortho-methoxycinnamic aldehyde, 3,5,6-trimethyl-3-cyclohexene carboxaldehyde, 3,7-dimethyl-2-methylene-6-octenal, phenoxyacetaldehyde, 5,9-dimethyl-4,8-decadienal, peony aldehyde (6,10-dimethyl-3-oxa-5,9-undecadien-1-al), hexahydro-4,7-methanoindan-1-carboxaldehyde, 2-methyl octanal, alpha-methyl-4-(1-methyl ethyl) benzene acetaldehyde, 6,6-dimethyl-2-norpinene-2-propionaldehyde, para methyl phenoxy acetaldehyde, 2-methyl-3-phenyl-2-propen-1-al, 3,5,5-trimethyl hexanal, Hexahydro-8,8-dimethyl-2-naphthaldehyde, 3-propyl-bicyclo[2.2.1]-hept-5-ene-2-carbaldehyde, 9-decenal, 3-methyl-5-phenyl-1-pentanal, methylnonyl acetaldehyde, 1-p-menthene-q-carboxaldehyde, citral, lilial and mixtures thereof. In one aspect of Applicants' invention perfume aldehydes are selected from the group consisting of citral, 1-decanal, benzaldehyde, florhydral® (3-(3-Isopropyl-phenyl)-butyraldehyde), 2,4-dimethyl-3-cyclohexen-1-carboxaldehyde; cis/trans-3,7-dimethyl-2,6-octadien-1-al; heliotropin; 2,4,6-trimethyl-3-cyclohexene-1-carboxaldehyde; 2,6-nonadienal; alpha-n-amyl cinnamic aldehyde, alpha-n-hexyl cinnamic aldehyde, P.T. Bucinal, lyral® (4-(4-hydroxy-4-methyl pentyl)-3-cyclohexene-1-carboxaldehyde), cymal, methyl nonyl acetaldehyde, trans-2-nonenal, lilial, trans-2-nonenal, and mixtures thereof.

Other suitable benefit agents include flavor ingredients including spices or flavor enhancers that contribute to the overall flavor perception of the product into which the benefit agent delivery system is incorporated. Pharmaceutical benefit agents including drugs. In one aspect of Applicants' invention a therapeutically acceptable amount of drug is empolyed. Biocontrol agents including biocides, antimicrobials, bactericides, fungicides, algaecides, mildewcides, disinfectants, sanitizer-like bleaches, antiseptics, insecticides, insect and/or moth repellant, vermicides, plant growth hormones, and the like. Antimicrobials including glutaraldehyde, cinnamaldehyde, and mixtures thereof. Typical insect and/or moth repellants such as citronellal, citral, N, N diethyl meta toluamide, Rotundial, 8-acetoxycarvotanacenone, and mixtures thereof. Other examples of insect and/or moth repellant for use as benefit agents herein are disclosed in U.S. Pat. Nos. 4,449,987, 4,693,890, 4,696,676, 4,933,371, 5,030,660, 5,196,200, and “Semio Activity of Flavor and Fragrance molecules on various Insect Species”, B. D. Mookherjee et al., published in Bioactive Volatile Compounds from Plants, ASC Symposium Series 525, R. Teranishi, R. G. Buttery, and H. Sugisawa, 1993, pp. 35-48. These publications are incorporated herein by reference.

Delivery System Forms

The benefit agent delivery systems herein may be based on the formation of a liquid or granular matrix. “Liquids” include fluids of density and viscosity that are conventional for liquids as well as gels and foams. Useful liquids may be aqueous or non-aqueous. Water is typically the major component of the delivery systems that are in aqueous liquid form. Conventional non-aqueous solvents may be used to form the matrix for liquid delivery systems in non-aqueous form. Liquid products, i.e., those containing 10% or greater of water or other solvents, are highly preferred.

Delivery systems in granular form can be fashioned from any type of solid-state material that comprises particles or granules ranging in size from 1 μm to 100 mm. Thus the granular matrix can include particles ranging from very fine powder to agglomerates or tablets. The granular matrix furthermore can comprise either inert or active ingredients, or both, with respect to the function of the product into which the delivery system is to be incorporated.

Most typically, the liquid or granular matrix used to form the delivery systems herein will comprise the matrix for the liquid or granular end product into which the benefit agent delivery system will be incorporated and made a part of. Thus, for example, liquid or granular detergent compositions for laundry or hard surface cleaning will frequently comprise the liquid or granular matrix into which the amine-based compounds and benefit agents described herein will be separately added to form the delivery systems of this invention.

Delivery System Preparation

It is an essential feature of the present invention that the amine compound and the benefit agent be added such that substantially no chemical reaction occurs between these materials prior to their contact with the liquid or granular matrix. For purposes of this invention, the amine-based compound and benefit agent are separately added to the system-forming matrix if the entire amounts of these components are combined with the matrix as discrete components. In particular, there must be essentially no chemical reaction between these two materials before they are combined with the matrix. Thus the amine compound and the benefit agent may be added to the matrix at separate times and/or from separate containers or from separate holding or delivery means. The amine compound and the benefit agent materials may even be mixed together prior to combination with the system-forming matrix so long as substantially no chemical reaction occurs between these materials prior to their contact with the system-forming matrix.

The benefit agent delivery system, especially in a granular form, can be prepared by simply admixing the amine-based compound and the benefit agent ketone and/or aldehyde under conditions which are sufficient to bring about combination, e.g., thorough admixture, of these components with the liquid or granular matrix. Frequently this admixing is carried out using high shear agitation. Temperatures of from about 40° C. to 65° C. may be utilized. Additional materials may also be added to the matrix in order to form the complete end product into which the delivery system is to be incorporated.

On a weight basis, the ratio of amine to benefit agent can vary widely, typically greater than about 1:5, more typically from about 1000:1 to about 1:1 for the two essential components. (amine compound and ketone/aldehyde benefit agent). In general, an excess of amine is desirable.

Adjunct Ingredients

Applicants' the various forms of Applicants' delivery system may contain adjunct ingredients including but not limited to water, surfactant, colorants and mixtures thereof.

Containers

Applicants' delivery may comprise one or more containers capable of containing the benefit agent and amine of the present invention in physical contact or sufficiently separate such that the benefit agent and amine are not in physical contact. Such one or more containers have separate compartments that may be empolyed to contain benefit agent and amine of the present invention in such a manner as to keep said benefit agent and amine sufficiently separate such that the benefit agent and amine are not in physical contact. One or more of said containers may comprise one or more spray dispensers capable of capable of dispensing said benefit agent and amine together or separately.

### EXAMPLE

| | Weight % | | | |
|---|---|---|---|---|
| Ingredients | 1 | 2 | 3 | 4 |
| Pro-fragrance component | | | | |
| Pro-fragrance¹ | 1.0 | — | — | — |
| Pro-fragrance² | 2.0 | — | — | — |
| Pro-fragrance³ | 2.0 | — | — | — |
| Free fragrance component | | | | |
| Damascone | 0.0001 | 0.015 | — | 0.01 |
| Melonal | 0.05 | 0.02 | — | — |
| Triplal ® | 0.01 | 0.03 | — | — |
| Beta-ionone | 0.01 | — | — | — |
| Additional free fragrance raw | 13.8 | 15.2 | 17.0 | 15.1 |
| materials containing at least | | | | |
| one aldehyde/ketone⁴ | | | | |
| Amine according to present | 0.09 | 0.5 | 1.5 | 0.03 |
| invention | | | | |
| Carrier⁵ | balance | balance | balance | balance |

¹Pro-fragrance which releases delta-damascone.
²Pro-fragrance which releases melonal.
³Pro-fragrance which releases triplal ®
⁴Conventional fragrance accord.
⁵Ethanol:water mixture (between 100:0 and 50:50).

1. A benefit delivery composition made by the process of combining:
a.) an amine material having a molecular weight of greater than 50 daltons, said amine material being selected from the group consisting of:
(i) non aromatic mono-amines comprising a primary or secondary amine;
(ii) non aromatic polyamines having at least 10% of their amine groups being selected from primary or secondary amines; and
(iii) and mixtures thereof;
b.) a benefit agent; and
c.) a liquid or granular matrix;
said process of combining being conducted such that substantially no chemical reaction occurs between said amine material and said benefit agent prior to their contact with said liquid or granular matrix.

2. The composition of claim 1 wherein said amine material comprises a primary amine moiety.

3. The benefit composition of claim 1 wherein said amine material comprises a hydroxyamine.

4. The composition of claim 3 wherein said amine material comprises a hydroxymonoamine.

5. The composition of claim 3 wherein said amine material comprises a mixture of amines comprising at least one of 2-hydroxyamine or 3-hydroxyamine.

6. The composition of claim 1 wherein said amine material comprises a polyamine.

7. The composition of claim 6 wherein at least about 15% of the amine groups of said polyamine are primary amine groups.

8. The composition of claim 1 wherein said benefit agent comprises a perfume material.

9. The composition of claim 8 wherein said perfume material comprises a perfume material selected from an aldehyde perfume, a ketone perfume or mixture thereof.

10. The composition of claim 9 wherein said benefit agent comprises a perfume material selected from the group consisting of Alpha Damascone, Delta Damascone, Iso Damascone, Carvone, Gamma-Methyl-Ionone, 1,2,3,4,5,6,7,8-octahydro-1,1,6,7-tetramethyl naphthalene-7-acetyl, 2,4,4,7-Tetramethyl-oct-6-en-3-one, Benzyl Acetone, Beta Damascone, Damascenone, methyl dihydrojasmonate, methyl cedrylone, hedione, citral, 1-decanal, benzaldehyde, 3-(3-Isopropyl-phenyl)-butyraldehyde, 2,4-dimethyl-3-cyclohexen-1-carboxaldehyde; cis/trans-3,7-dimethyl-2,6-octadien-1-al; heliotropin; 2,4,6-trimethyl-3-cyclohexene-1-carboxaldehyde; 2,6-nonadienal; alpha-n-amyl cinnamic aldehyde, alpha-n-hexyl cinnamic aldehyde, 4-(4-hydroxy-4-methyl pentyl)-3-cyclohexene-1-carboxaldehyde, cymal, methyl nonyl acetaldehyde, trans-2-nonenal, lilial, trans-2-nonenal, 2,4-dimethyl-3-cyclohexene-1-carboxaldehyde, 2,6-Dimethyl-5-heptenal and mixtures thereof.

11. The composition of claim 9 wherein said amine material comprises a hydroxyamine.

12. The composition of claim 11 wherein said amine material comprises at least one of 2-hydroxyamine or 3-hydroxyamine.

13. A method of delivering a benefit agent to a substrate, said method comprising applying the composition of claim 1, in neat form, to a substrate and then optionally rinsing and/or washing said substrate.
